(19) United States
(12) Patent Application Publication
(10) Pub. No.: US 2022/0040679 A1
(43) Pub. Date: Feb. 10, 2022

(54) Catalyst Compositions and Methods for Producing Long-Chain Hydrocarbon Molecules

(71) Applicant: BEIJING GUANGHE NEW ENERGY TECHNOLOGY CO., LTD., Beijing (CN)

(72) Inventor: Cong Wang, Beijing (CN)

(73) Assignee: BEIJING GUANGHE NEW ENERGY TECHNOLOGY CO., LTD., Beijing (CN)

(21) Appl. No.: 17/416,738

(22) PCT Filed: Dec. 20, 2018
(86) PCT No.: PCT/CN2018/122319

§ 371 Date: Jun. 21, 2021

(51) Int. Cl. B01J 23/89; B01J 35/08; B01J 35/02; B01J 23/75; B01J 23/745; B01J 23/34; B01J 23/656; C07C 2/24

(52) U.S. Cl. CPC B01J 23/8913; B01J 35/08; B01J 35/023; B01J 23/75; B01J 23/745; B01J 23/8966; C07C 2523/889; B01J 23/6562; C07C 2/24; C07C 2523/75; C07C 2523/89; C07C 2521/06; C07C 2523/34; B01J 23/34

(57) ABSTRACT

Provided is a nanostructure catalyst composition and a method for producing organic molecules having at least two carbon atoms chained together by the reaction of a hydrogen-containing source, a carbon-containing source and an optional nitrogen-containing source. Composition of the nanostructure catalyst affects the solar-to-chemical efficiency, active lifetime and reaction product of the artificial photosynthesis reaction.

### FIELD OF THE INVENTION

The invention generally relates to carbon dioxide sequestration and renewable energy. More particularly, the invention relates to catalyst compositions and methods for producing long-chain hydrocarbon molecules.

### BACKGROUND

Carbon emissions contribute to climate change, which can have serious consequences for humans and the environment. Many endeavors were made in order to capture, utilize, and non-atmospheric sequester the carbon dioxide emitted from fossil fuel-fired electric power plants and industrial plants. Some technologies have shown great promise in this area but are still long way from demonstrating on a commercial scale. It is a priority to establish technical, environmental, and economic feasibility of large-scale capture and disposal of carbon dioxide from industrial plants.

Conventional approach for carbon dioxide sequestration is generally directed to artificial photosynthesis using sunlight as the energy source. Most efforts so far are devoted for the development of catalysts, but the solar-to-chemical efficiencies are typically 1 or 2 orders of magnitude lower than natural photosynthesis, which is lacking of efficiency for industrial application.

Besides, there has been growing achievements in the past decades in the field of solar-based technologies which produce either electricity or hydrogen. However, for artificial photosynthesis reactions, the product thereof is not readily controllable, which is also undesirable for industrial purposes.

### SUMMARY OF THE INVENTION

Herein, the inventors have demonstrated a novel artificial carbon sequestration technology which provided a unique catalyst composition and method for producing long-chain organic molecules by utilizing CO or CO₂ from industrial flue gas or atmosphere.

One aspect of the present invention relates to a nanostructure catalyst composition, comprising:

at least one plasmonic provider; and

at least one catalytic property provider, wherein

the plasmonic provider and the catalytic property provider are in contact with each other or have a distance of less than 200 nm apart from each other, preferably of less than about 100 nm apart from each other, and

the plasmonic provider is about 0.1%-30% by mole of a total mole of the plasmonic provider and the catalytic property provider.

In certain embodiments, the plasmonic provider is about 0.1%-10% by mole of a total mole of the plasmonic provider and the catalytic property provider, preferably about 3%-8% by mole, about 4%-6% by mole. The solar-to-chemical efficiency of such nanostructure catalyst composition shall be more than 10%.

In certain embodiments, the plasmonic provider is about 10%-30% by mole of a total mole of the plasmonic provider and the catalytic property provider, preferably about 15%-25% by mole, about 18%-20% by mole. The active lifetime of the nanostructure catalyst composition shall be more than 10 days.

In preferred embodiments, the plasmonic provider is selected from the group consisting of Co, Mn, Fe, Al, Ag, Au, Pt, Cu, Ni, Zn, Ti, C or any combination thereof. In specific embodiments, the plasmonic provider comprises 10%-100% by mole, preferably 90%-100% by mole of Co, Mn, Fe, Al, Ag, Au, Pt, Cu, Ni and/or Zn, preferably of Co, Mn, Fe, Al, Cu, Ni and/or Zn, and less than 10% by mole of Ti and/or C, relative to a total mole of the plasmonic provider.

In preferred embodiments, the catalytic property provider is selected from the group consisting of Co, Mn, Ag, Fe, Ru, Rh, Pd, Os, Ir, La, Ce, Cu, Ni, Ti, oxides thereof, hydroxides thereof, chlorides thereof, carbonates thereof, bicarbonates thereof, C, or any combination thereof. In specific embodiments, the catalytic property provider comprises 10%-100% by mole, preferably 90%-100% by mole of Co, Mn, Fe, Ni, Cu, Ti, oxides thereof, chlorides thereof, carbonates thereof, and/or bicarbonates thereof, less than 10% by mole of Ru, Rh, Pd, Os, Ir, La, Ce, oxides thereof, chlorides thereof, carbonates thereof, and/or bicarbonates thereof, and less than 10% by mole of C, relative to a total mole of the catalytic property provider.

In preferred embodiments, the nanostructure catalyst composition comprises one or more of the following combination of elements: Co/Fe/C; Co/Ti/Au; Co/Ti/Ag; Co/Au; and Co/Ag. Particularly, in the combination of Co/Fe/C, Co is about 0.1%-10% by mole of a total mole of Co, Fe and C, preferably about 3%-8% by mole, about 4%-6% by mole; in the combination of Co/Ti/Au, Au is about 0.1%-30% by mole of a total mole of Co, Ti and Au, preferably about 0.1%-10% by mole, about 3%-8% by mole, about 4%-6% by mole, or preferably about 10%-30% by mole, about 15%-25% by mole, about 18%-20% by mole; in the combination of Co/Ti/Ag, Ag is about 10%-30% by mole of a total mole of Co, Ti and Ag, preferably about 15%-25% by mole, about 18%-20% by mole; in the combination of Co/Au, Au is about 0.1%-10% by mole of a total mole of Co and Au, preferably about 3%-8% by mole, about 4%-6% by mole; and in the combination of Co/Ag, Ag is about 0.1%-10% by mole of a total mole of Co and Ag, preferably about 3%-8% by mole, about 4%-6% by mole.

In alternative embodiments, the nanostructure catalyst composition comprises 10% or less or 90% or more by mole of C.

In certain embodiments, the nanostructures of the nanostructure catalyst composition each independently is from about 1 nm to about 3000 nm in length, width or height, preferably from about 100 nm to about 3000 nm, from about 500 nm to about 2500 nm, or from 1000 nm to about 2000 nm in length, and/or from about 1 nm to about 1000 nm, from about 100 nm to about 800 nm, from about 200 nm to about 500 nm in width or height, or the nanostructures each independently has an aspect ratio of from about 1 to about 20, from about 1 to about 10, or from about 2 to about 8.

In certain embodiments, the nanostructures each independently has a shape of spherical, spike, flake, needle, grass, cylindrical, polyhedral, 3D cone, cuboidal, sheet, hemispherical, irregular 3D shape, porous structure or any combinations thereof.

In certain embodiments, multiple nanostructures are arranged in a patterned configuration, in a plurality of layers, on a substrate, or randomly dispersed in a medium.

Another aspect of the present invention is a method for producing organic molecules having at least two carbon atoms chained together by the reaction of a hydrogen-containing source, a carbon-containing source and an optional nitrogen-containing source in the presence of a nanostructure catalyst composition according to the first aspect of the present invention.

In certain embodiments, the organic molecules comprise saturated, unsaturated and aromatic hydrocarbons, carbohydrates, amino acids, polymers, or any combination thereof.

In specific embodiments, the organic molecules comprise linear saturated hydrocarbons having 20 carbon atoms or less when the catalytic property provider is selected from the group consisting of Co, Mn or combination thereof.

In specific embodiments, the organic molecules comprise linear saturated hydrocarbons having 20 carbon atoms or more when the catalytic property provider is Fe.

In specific embodiments, the organic molecules comprise linear saturated hydrocarbons having 3 carbon atoms or less when the catalytic property provider is selected from the group consisting of Ni, Cu or combination thereof.

In specific embodiments, the organic molecules comprise linear and branched, saturated and unsaturated hydrocarbons having 5 to 10 carbon atoms when the catalytic property provider is selected from the group consisting of Ru, Rh, Pd, Os, Ir, La, Ce or any combination thereof.

In certain embodiments, the reaction is initiated by light irradiation or initiated by heat.

In certain embodiments, the reaction is progressed under a temperature between about 50° C. and about 800° C., preferably between about 50° C. and about 500° C., between about 80° C. and about 300° C., between about 120° C. and about 200° C.

In preferred embodiments, the carbon-containing source comprises CO₂ or CO, and the hydrogen-containing source comprises water.

### BRIEF DESCRIPTION OF THE DRAWINGS

FIG. 1. GC-MS chromatogram of reaction products of Experiment 11 of Example 2.

FIG. 2. GC-MS chromatogram of reaction products of Experiment 12 of Example 2.

FIG. 3. GC-MS chromatogram of reaction products of Experiment 13 of Example 2

FIG. 4. GC-MS chromatogram of reaction products of Experiment 14 of Example 2.

### DETAILED DESCRIPTION

The invention has demonstrated that, surprisingly and unexpectedly, solar-to-chemical efficiency and active lifetime of artificial photosynthesis reaction is greatly affected by the composition of the nanostructure catalyst, and control of product composition of artificial photosynthesis is also achievable by providing different nanostructure catalyst compositions.

Before further description of the present invention, certain terms employed in the specification, examples and appended claims are defined in the following section. The definitions listed herein should be read in light of the remainder of the disclosure and understood as by a person of skill in the art. Unless defined otherwise, all technical and scientific terms used herein have the same meaning as commonly understood by a person of ordinary skill in the art to which this invention belongs.

Nanostructure Catalyst

Nanostructure catalyst is used in the reaction of the present invention for producing organic molecules.

Without wishing to be bound by theory, the nanostructure catalyst of the present invention interacts with the raw materials of the reaction to reduce the activation energy of the reaction so as to initiate the reaction by utilizing solar or thermal energy.

The nanostructure catalyst composition of the present invention comprises two components. One component is plasmonic provider, and the other component is catalytic property provider. Plasmonic provider provides surface plasmon resonance enhancement to the localized field on catalysts when excited by electromagnetic irradiation. Catalytic property provider provides catalytic property to the reaction that produces hydrocarbons.

In the nanostructure catalyst composition, the plasmonic provider and the catalytic property provider are in contact with each other or have have a distance of less than 200 nm apart from each other, preferably of less than about 100 nm apart from each other. If the distance between the plasmonic provider and the catalytic property provider are outside aforementioned range, two kinds of providers cannot exert the effect in cooperation with each other, thus cannot catalyze the photosynthesis reaction.

In the nanostructure catalyst composition, the plasmonic provider is about 0.1%-30% by mole of a total mole of the plasmonic provider and the catalytic property provider for achieving higher solar-to-chemical efficiency and longer active lifetime of the catalyst. More specifically, when the plasmonic provider is about 0.1%-10% of the total mole, preferably about 3%-8%, about 4%-6%, the solar-to-chemical efficiency of the nanoparticle catalyst composition is more than 10%, 12%, 15%, 20%, or even higher. When the plasmonic provider is about 10%-30% of the total mole, preferably about 15%-25%, about 18%-20%, the active lifetime of the nanoparticle catalyst composition is more than 10 days, 15 days, 20 days, 30 days, or even longer. However, when further increasing the molar ratio of the plasmonic provider to more than 30%, the solar-to-chemical efficiency gradually decreases and is not suitable for industrial uses.

Plasmonic provider is a conductor whose real part of its dielectric constant is negative. It can be a pure substance or a mixture, and the composing element is one or more selected from Co, Mn, Fe, Al, Ag, Au, Pt, Cu, Ni, Zn, Ti, C or any combination thereof. Different plasmonic providers have different plasmon enhancement strength and active lifetime. Preferably, the plasmonic provider rises 10%-100% by mole, preferably 90%-100% by mole of Co, Mn, Fe, Al, Ag, Au, Pt, Cu, Ni and/or Zn, preferably of Co, Mn, Fe, Al, Cu, Ni and/or Zn, and less than 10% by mole of Ti and/or C, relative to a total mole of the plasmonic provider.

Catalytic property provider can be a pure substance or a mixture, and the composing element is one or more selected from Co, Mn, Ag, Fe, Ru, Rh, Pd, Os, Ir, La, Ce, Cu, Ni, Ti, oxides thereof, hydroxides thereof, chlorides thereof, carbonates thereof, bicarbonates thereof, C, or any combination thereof. Different catalytic property providers also have different catalytic strength and active lifetime. Preferably, the catalytic property provider comprises 10%-100% by mole, preferably 90%-100% by mole of Co, Mn, Fe, Ni, Cu and/or Ti species, less than 10% by mole of Ru, Rh, Pd, Os, Ir, La and/or Ce species, and less than 10% by mole of C. The term “species” of a chemical element used herein refer to elemental substance or compounds of the element. For example, “Co species” include elemental Co, CoO, CoCl₂, CoCO₃, as well as other compounds comprising Co.

It is an unexpected effect of the present invention that the product of the artificial photosynthesis reaction catalyzed by the nanostructure catalyst composition can be controlled by the elemental composition of the catalytic property provider. More specifically, Co and Mn lead to relative shorter chain hydrocarbon (carbon #<20); Fe leads to relative longer chain hydrocarbon (carbon #>20); Ni and Cu leads to even shorter chain hydrocarbon (carbon #<3); Ru, Rh, Pd, Os, Ir, La and Ce lead to unsaturated or branched hydrocarbon in a carbon number range, such as (5<carbon #<10).

In most preferred embodiments of the present invention, the nanoparticle catalyst composition comprises one or more of the following combination of elements: Co/Fe/C; Co/Ti/Au; Co/Ti/Ag; Co/Au; and Co/Ag. Such compositions may achieve high solar-to-chemical efficiency, long active lifetime and are cost effective for industrial application. For example, in the combination of Co/Fe/C, Co is about 0.1%-10% by mole of a total mole of Co, Fe and C, preferably about 3%-8% by mole, about 4%-6% by mole; in the combination of Co/Ti/Au, Au is about 0.1%-30% by mole of a total mole of Co, Ti and Au, preferably about 0.1%-10% by mole, about 3%-8% by mole, about 4%-6% by mole, or preferably about 10%-30% by mole, about 15%-25% by mole, about 18%-20% by mole; in the combination of Co/Ti/Ag, Ag is about 10%-30% by mole of a total mole of Co, Ti and Ag, preferably about 15%-25% by mole, about 18%-20% by mole; in the combination of Co/Au, Au is about 0.1%-10% by mole of a total mole of Co and Au, preferably about 3%-8% by mole, about 4%-6% by mole; and in the combination of Co/Ag, Ag is about 0.1%-10% by mole of a total mole of Co and Ag, preferably about 3%-8% by mole, about 4%-6% by mole.

In alternative embodiments of the present invention, the nanostructure catalyst composition comprises 10% or less or 90% or more by mole of C. Typically, C is less than 10% by mole in the nanostructure catalyst composition for optimizing solar-to-chemical efficiency. Without wishing to be bound by theory, it is found in the present application that when C is more than 90% by mole in the nanostructure catalyst composition, active lifetime of the catalyst is greatly increased to more than 10 days, and even more than 1 year. However, a mole ratio of C between 10% and 90% is not suitable for the catalyst composition of the present invention. C can be provided in the forms of nanoparticle of graphite, graphene, carbon nanotube, etc.

Nanostructure

The term “nanostructure” used herein refers to a structure having at least one dimension within nanometer range, i.e. 1 nm to 1000 nm in at least one of its length, width, and height. Nanostructure can have one dimension which exceeds 1000 nm, for example, have a length in micrometer range such as 1 micron to 5 micron. In certain cases, tubes and fibers with only two dimensions within nanometer range are also considered as nanostructures. Material of nanostructure may exhibit size-related properties that differ significantly from those observed in bulk materials.

The nanostructure of the present invention each independently is from about 1 nm to about 3000 nm in length, width or height. The length thereof is preferably from about 100 nm to about 3000 nm, more preferably from about 500 nm to about 2500 nm, yet more preferably from 1000 nm to about 2000 nm. The width or height thereof is preferably from about 1 nm to about 1000 nm, more preferably from about 100 nm to about 800 nm, yet more preferably from about 200 nm to about 500 nm.

The nanostructure of the present invention each independently has an aspect ratio (i.e., length to width/height ratio) of from about 1 to about 20, from about 1 to about 10, or from about 2 to about 8. The nanostructure of the present invention can also have a relatively low aspect ratio such as from about 1 to about 2.

The nanostructure of the present invention each independently has a shape of spherical, spike, flake, needle, grass, cylindrical, polyhedral, 3D cone, cuboidal, sheet, hemispherical, irregular 3D shape, porous structure or any combinations thereof.

Multiple nanostructures of the present invention can be arranged in a patterned configuration, in a plurality of layers, on a substrate, or randomly dispersed in a medium. For example, nanostructures may be bound to a substrate. In such case, the nanostructures are generally not aggregated together, but rather, pack in an orderly fashion. Alternatively, multiple nanostructures can be dispersed in a fluid medium, in which each nanostructure is free to move with respect to any other nanostructures.

For example, the nanostructure may take a spike or grass-like geometry. Alternatively, the nanostructure has a flake-like geometry having a relatively thin thickness. Preferably, the nanostructure takes on a configuration of nanoforest, nanograss and/or nanoflake. The nanostructure may have a relatively high aspect ratio, such nanostructure may adopt a configuration of nano-spike, nano-flake or nano-needle. The aspect ratio can be from about 1 to about 20, from about 1 to about 10, or from about 2 to about 8. Preferably, the length of the nanostructure can be from about 100 nm to about 3000 nm, from about 500 nm to about 2500 nm, or from 1000 nm to about 2000 nm; the width or height can be from about 1 nm to about 1000 nm, from about 100 nm to about 800 nm, or from about 200 nm to about 500 nm.

The nanostructure may be bound to a substrate. Accordingly, the nanostructures are generally not aggregated together, but rather, packed in an orderly fashion. The substrate can be formed of a metal or a polymeric material (e.g., polyimide, PTFE, polyester, polyethylene, polypropylene, polystyrene, polyacrylonitrile, etc.).

In some embodiments, the nanostructure can comprise a metal oxide coating formed spontaneously or intentionally onto the metal portion. In certain embodiments, a nanostructure can be formed into two or more layers with different element compositions in each layer.

In other examples, the nanostructures take the shape of spheres, cylinders, polyhedrons, 3D cones, cuboids, sheets, hemisphere, irregular 3D shapes, porous structure and any combinations thereof. The nanostructures each independently has a length, width and height from about 1 nm to about 1000 nm, preferably from about 100 nm to about 800 nm, or from about 200 nm to about 500 nm. The plasmonic provider and the catalytic property provider can be randomly mixed, or regularly mixed. The plasmonic provider and the catalytic property provider are in contact with each other or apart from each other by a distance less than about 200 nm, preferably less than about 100 nm. In preferred embodiments, the two components are provided in one nano structure, i.e. an alloy of two or more chemical elements.

In addition, the nanostructure catalyst composition functions in various states, such as dispersed, congregated, or attached/grew on the surface of other materials. In preferred embodiments, the nanostructures are dispersed in a medium, in which the medium is preferably a reactant of the reaction, such as water.

Method for Producing Organic Molecules

A method for producing organic molecules having at least two carbon atoms chained together is provided in the present invention. The nanostructure catalyst composition as described above is used in the method to control solar-to-chemical efficiency, active lifetime and product composition of the artificial photosynthesis reaction.

The method comprises the reaction of at least a hydrogen-containing source, a carbon-containing source, and an optional nitrogen-containing source in the presence of the nanostructure catalyst composition. The reaction may be initiated by light irradiation or by heat.

Reaction Initiation by Light Irradiation

The light irradiation initiates a reaction of the carbon-containing source and the hydrogen-containing source with the catalysis of the plasmonic nanostructure catalyst. Within a certain temperature range, raising the temperature leads to a higher yield of the hydrocarbon molecule products.

The light irradiation step is performed under a temperature between about 20° C. to about 800° C., about 30° C. to about 300° C., about 50° C. to about 250° C., about 70° C. to about 200° C., about 80° C. to about 180° C., about 100° C. to about 150° C., about 110° C. to about 130° C., etc. In order to obtain fuel-like hydrocarbon molecules, the temperature is preferred to be between about 70° C. to about 200° C. Solar-to-chemical efficiency is more than 10% at above mentioned temperatures.

The light irradiation simulates the wavelength composition and intensity of sunlight, therefore it may raise the temperature of the irradiated catalysts and reactants mixture. When the irradiation intensity reaches a certain level, the temperature of the plasmonic nanostructure catalyst, the carbon-containing source and the hydrogen-containing source is solely raised by the light irradiation.

Reaction Initiation by Heat

On contrary to the above approach of simulating photosynthesis by using light energy as the energy input for the endothermic reaction, the artificial photosynthesis reaction can be initiated by heat in a dark environment. After the reaction is initiated, the reaction can continue to progress in the dark environment with the thermal energy of a heat source.

The term “heat” used herein refers to thermal energy transferred from one system to another as a result of thermal interactions. Heat may be transferred externally into the reaction with an external heat source. Alternatively, heat may be inherently carried by one component of the reaction so as to be transferred to other components involved in the reaction. In other words, the one component that inherently carries heat is an internal heat source.

In certain embodiments, the heat which initiates the reaction is input externally into the reaction, or is inherently carried by one or more of the hydrogen-containing source, the carbon-containing source and the optional nitrogen-containing source. Preferably, the heat is inherently carried by the carbon-containing source.

In preferred embodiments, the temperature of the catalyst, the carbon-containing source and the hydrogen-containing source is solely raised by a heat source during the reaction. That is to say, temperature of the reaction system is not raised by another energy source, such as light source.

The term “light” used herein refers to electromagnetic wave having a wavelength from about 250 nm to about 1000 nm. In other words, light refers to the irradiance of visible light.

The term “dark environment” used herein refers to an environment with substantially no incoming or incident light. For example, a dark environment is an environment having no light sources irradiating therein that has a radiation intensity capable of initiating a photosynthesis reaction. Moreover, the dark environment has substantially no incoming or incident light transmitting through the boundary between the dark environment and its surrounding.

Alternatively, in a dark environment with substantially no incoming or incident light, the light irradiation intensity within the environment is not capable of increasing the temperature of the reaction system, which means the irradiation intensity is close to zero.

Specifically, the light radiation intensity at any location within a dark environment is below 1 W/cm², preferably below 1 mW/cm², and most preferably below 1 μW/cm².

For example, the reaction can be initiated by heat in a dark environment as perceived by a skilled person in the art, such as a curtained container, a closed pipeline or a dark room. After initiation, the reaction continues to progress in a dark environment.

After initiation of the reaction either by light irradiation or by heat, the reaction is progressed under a temperature between about 50° C. and about 800° C., preferably between about 50° C. and about 500° C., between about 80° C. and about 300° C., between about 120° C. and about 200° C. The reaction may progress under light or in a dark environment with substantially no light as long as a suitable temperature is maintained for the reaction. It should be understood that the production of organic compounds in the presence of the nanostructure catalyst composition is capable to progress under any light intensity.

The reaction period is not particularly limited in the present invention as long as the organic molecules are produced. The reaction can be a continuous reaction or an intermittent reaction. In other words, the reaction can be repeatedly initiated and terminated according to actual needs. With a well-established apparatus, the reaction is continuously performed with a continuous feed of light or heat and reaction materials.

Reaction Materials

In the reaction of the present invention, the reaction materials comprise a hydrogen-containing source, a carbon-containing source and an optional nitrogen-containing source.

The carbon-containing source is selected from the group consisting of CO₂, CO, C₁₋₄ hydrocarbons, C₁₋₄ alcohols, synthesis gas, bicarbonate salts and any combination thereof, or air, industrial flue gas, exhausts or emissions comprising one or more of these carbon-containing sources. The preferable carbon-containing source is CO₂ and CO.

The hydrogen-containing source is selected from the group consisting of water, H₂, C₁₋₄ hydrocarbons, C₁₋₄ alcohols and any combination thereof in liquid or gaseous phase, or waste water, industrial flue gas, exhausts or emissions comprising one or more of these hydrogen-containing sources. The preferable hydrogen-containing source is water.

The nitrogen-containing source is selected from the group consisting of N₂, air, ammonia, nitrogen oxides, nitro compounds, C₁₋₄ amines and any combination thereof in liquid or gaseous phase, or air, industrial flue gas, exhausts or emissions comprising one or more of these nitrogen-containing sources. The preferable nitrogen-containing source is ammonia and air.

For the purposes of recycling and treatment of industrial wastes, waster water, flue gas, combustion emission and automobile exhaust which contains the hydrogen-containing source, the carbon-containing source and the nitrogen-containing source can be used as the reaction material of the present invention. It is notable that the latent heat contained in the industrial waste is also utilized by the reaction, which is particularly useful in recycling the material and thermal energy contained in the industrial waste.

Reaction Products

The reaction of the present invention is capable to produce organic molecules having at least two carbon atoms chained together. The organic molecules comprise saturated, unsaturated and aromatic hydrocarbons, carbohydrates, amino acids, polymers, or a combination thereof.

According to one advantage of the present invention, when catalytic property provider is selected from the group consisting of Co, Mn and combination thereof, the reaction product mainly comprises linear saturated hydrocarbons having 20 carbon atoms or less;

when the catalytic property provider is Fe, the reaction product mainly comprises linear saturated hydrocarbons having 20 carbon atoms or more;

when the catalytic property provider is selected from the group consisting of Ni, Cu and combination thereof, the reaction product mainly comprises linear saturated hydrocarbons having 3 carbon atoms or less; and

when the catalytic property provider is selected from the group consisting of Ru, Rh, Pd, Os, Ir, La, Ce and combination thereof, the reaction product mainly comprises linear and branched, saturated and unsaturated hydrocarbons having 5 to 10 carbon atoms

In further embodiments, when a nitrogen-containing source is included in the reaction, the product can be amino acids or other polymers having nitrogen atoms in the structure.

Aspects of the method of the present invention has been detailed described in the foregoing contents, which will be more apparent to the skilled in the art upon reading the following preferred embodiments of the present invention.

### EXAMPLES

### Example 1. Ratio Between Plasmonic Provider (PP) and Catalytic Property Provider (CPP)

2 g of nanostructure catalyst composition according to Table 1 was loaded in a glass reactor (20 ml), and 350 mg of distilled water were added into the reactor to immerse the catalyst. The catalysts have a shape of nearly spherical nanoparticles having diameters of 200 nm. The reactor was then filled with CO₂ and was sealed. For experiments longer than 1 day, production vapor was removed from the reactor per day, and water and CO₂ were added to maintain the initial condition (350 mg water, filled with CO₂). The reactor was irradiated by solar simulator (about 1000-1200 W/m²) at a temperature of 120° C. or was heated in a dark oven at a temperature of 120° C. The irradiation or heating is maintained during the entire reaction duration.

After reaction for a certain duration according to Table 1, the reactor was removed from irradiation or heating and cooled to room temperature. Approximately 3 mL of dichloromethane (DCM, HPLC grade, 99.9%) was injected into each reactor and shaken for ˜10 min in order to extract non-volatile organic products. The DCM extracts were then analyzed by GC-MS (Agilent and Bruker).

TABLE 1
catalyst composition and reaction efficiency

| Ex | PP (composition | CPP (composition | Reaction | Light | |
|---|---|---|---|---|---|
| # | and mole ratio) | and mole ratio) | time | or heat | Efficiency |
| 1 | Co (10%) | Fe/C (90%) | 8 hrs | Light | ~10% |
| 2 | Cu (10%) | Fe/C (90%) | 8 hrs | Light | ~1% |
| 3 | Au (10%) | Ti/Co (90%) | 8 hrs | Light | ~10% |
| 4 | Au (10%) | Ti/Co (90%) | >10 days | Light | ~1% |
| 5 | Au (20%) | Ti/Co (80%) | 1-15 days | Light | ~5% |
| 6 | Au (10%) | Co (90%) | 8 hrs | Heat | ~15% |
| 7 | Au (50%) | Co (50%) | 8 hrs | Heat | ~0.1% |
| 8 | Ag (10%) | Co (90%) | 8 hrs | Heat | ~11% |
| 9 | Ag (20%) | Ti/Co (80%) | 1-15 days | Light | ~5% |
| 10 | Mn (10%) | C (90%) | 1-15 days | Light | ~5% |

From above results, it is clear that when the mole ratio of PP is 10% or less, higher solar-to-chemical efficiency is achieved; when the mole ratio of PP is higher than 10% and lower than 30%, longer active lifetime is achieved.

For instance, as shown in Experiment #3, #4 and #5, when the ration of Au (PP) is 10%, the efficiency is about 10% (#3) at 8 hours; but during 10 days of reaction, the efficiency gradually decreases, and reaches about 1% after 10 days (#4); when the ration of Au (PP) is 20%, the efficiency is stable at ˜5% for >10 days (#5). However, when the mole ratio of PP is 50% (#7), the efficiency is not suitable for practical uses.

Further experiments were also conducted by changing the mole ratio of PP and CPP without changing the respective element composition. For example, for Experiment #1-4, 6, 8, and 10, when the PP part reduces to about 8%, 6%, 5% or 3%, the reaction will not be changed over all; and the efficiency will also be maintained at nearly same levels. For Experiment #5 and 9, when the PP part changes to about 12%, 15%, 18%, 25% or 30%, the reaction will not be changed over all; and lifetime will also be maintained at nearly same levels.

### Example 2. Ratio Between Different Elements of Catalytic Property Provider (CPP)

Additional experiments were conducted according to the method of Example 1 and the nanostructure catalyst composition according to Table 2 was used. Instead of total mass of reaction product, product composition was investigated from the GC-MS chromatogram.

TABLE 2
element composition of CPP and reaction product

| | CPP (composition | | Light or | |
|---|---|---|---|---|
| Ex # | and mole ratio) | Reaction time | heat | Product composition |
| 11 | Co/Mn (50%/50%) | 8 hrs | Light | carbon # <20 |
| 12 | Fe (100%) | 10 hrs | Light | carbon # >20 |
| 13 | Ni/Cu (50%/50%) | 10 hrs | Heat | carbon # <3 |
| 14 | Ru/Mn (5%/95%) | 20 hrs | Light | 5 < carbon # <10, |
| | | | | unsaturated, |
| | | | | branched |

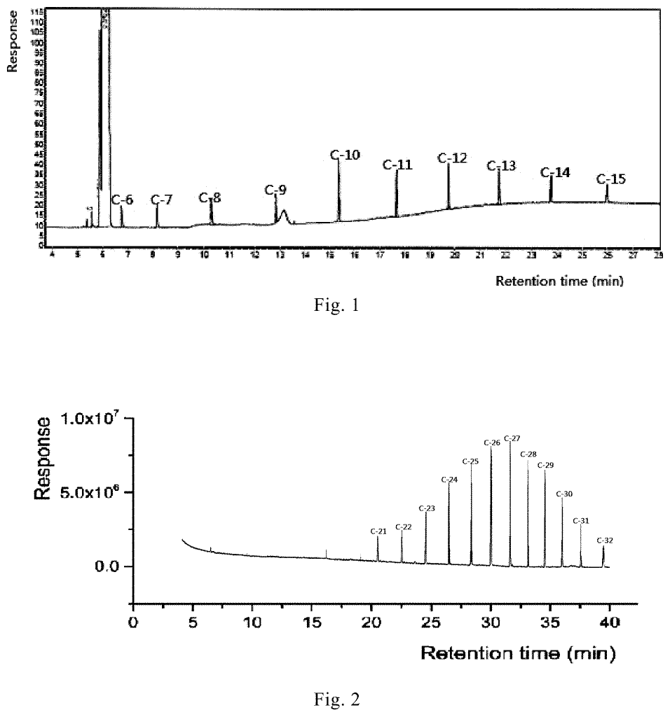

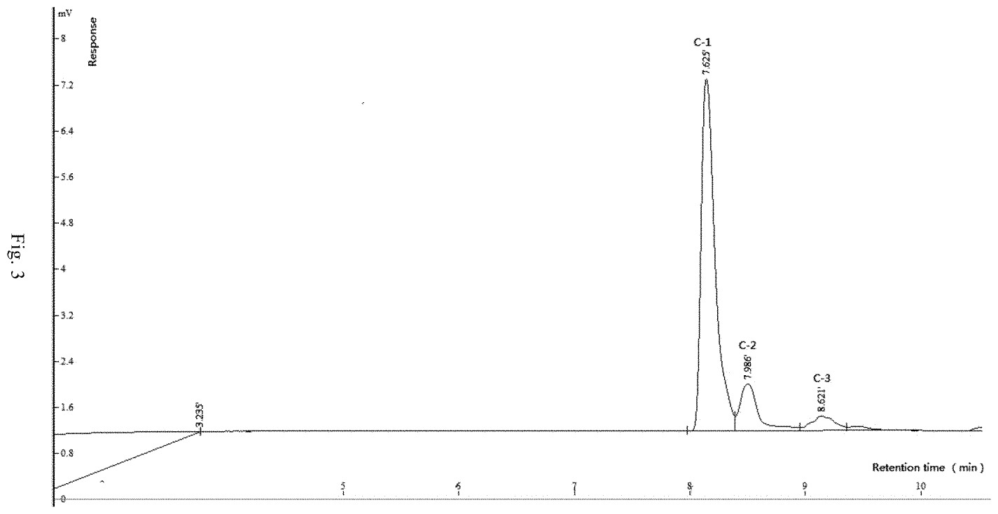

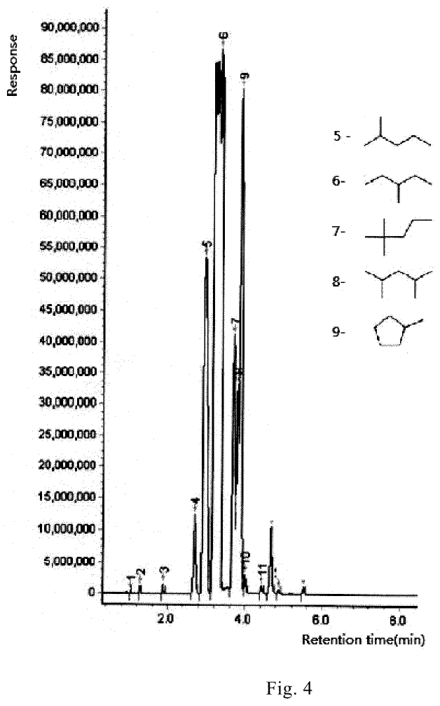

GC-MS chromatograms of the reaction products of Examples 11-14 are shown in FIGS. 1-4.

From above results, it is clear that modification to the elemental composition of the CPP can control the product composition of the reaction. Moreover, similar results are demonstrated when replacing Ru in Experiment #14 with other elements such as Rh, Pd, Os, Ir, La, and/or Ce, as long as such elements comprise less than 10% by mole of CPP.

In this specification and the appended claims, the singular forms “a”, “an”, and “the” include plural reference, unless the context clearly indicates otherwise. Unless defined otherwise, all technical and scientific terms used herein have the same meaning as commonly understood to one of ordinary skill in the art. Methods recited herein may be carried out in any order that is logically possible, in addition to a particular order disclosed.

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A nanostructure catalyst composition, comprising:
at least one plasmonic provider; and
at least one catalytic property provider, wherein
the plasmonic provider and the catalytic property provider are in contact with each other or have a distance of less than 200 nm apart from each other, and
the plasmonic provider is about 0.1%-30% by mole of a total mole of the plasmonic provider and the catalytic property provider.

2. The nanostructure catalyst composition of claim 1, wherein
the plasmonic provider is about 0.1%-10% by mole of a total mole of the plasmonic provider and the catalytic property provider, about 4%-6% by mole.

3. The nanostructure catalyst composition of claim 1, wherein
the plasmonic provider is about 10%-30% by mole of a total mole of the plasmonic provider and the catalytic property provider, about 18%-20% by mole.

4. The nanostructure catalyst composition of claim 1, wherein
the plasmonic provider is selected from the group consisting of Co, Mn, Fe, Al, Ag, Au, Pt, Cu, Ni, Zn, Ti, C or any combination thereof.

5. The nanostructure catalyst composition of claim 4, wherein
the plasmonic provider comprises 10%-100% by mole, of Co, Mn, Fe, Al, Ag, Au, Pt, Cu, Ni and/or Zn, and less than 10% by mole of Ti and/or C, relative to a total mole of the plasmonic provider.

6. The nanostructure catalyst composition of claim 1, wherein
the catalytic property provider is selected from the group consisting of Co, Mn, Ag, Fe, Ru, Rh, Pd, Os, Ir, La, Ce, Cu, Ni, Ti, oxides thereof, hydroxides thereof, chlorides thereof, carbonates thereof, bicarbonates thereof, C, or any combination thereof.

7. The nanostructure catalyst composition of claim 6, wherein
the catalytic property provider comprises 10%-100% by mole of Co, Mn, Fe, Ni, Cu, Ti, oxides thereof, chlorides thereof, carbonates thereof, and/or bicarbonates thereof, less than 10% by mole of Ru, Rh, Pd, Os, Ir, La, Ce, oxides thereof, chlorides thereof, carbonates thereof, and/or bicarbonates thereof, and less than 10% by mole of C, relative to a total mole of the catalytic property provider.

8. The nanostructure catalyst composition of claim 1, wherein
the nanostructure catalyst composition comprises one or more of the following combination of elements: Co/Fe/C; Co/Ti/Au; Co/Ti/Ag; Co/Au; and Co/Ag.

9. The nanostructure catalyst composition of claim 8, wherein
in the combination of Co/Fe/C, Co is about 0.1%-10% by mole of a total mole of Co, Fe and C;
in the combination of Co/Ti/Au, Au is about 0.1%-30% by mole of a total mole of Co, Ti and Au;
in the combination of Co/Ti/Ag, Ag is about 10%-30% by mole of a total mole of Co, Ti and Ag;
in the combination of Co/Au, Au is about 0.1%-10% by mole of a total mole of Co and Au; and
in the combination of Co/Ag, Ag is about 0.1%-10% by mole of a total mole of Co and Ag.

10. The nanostructure catalyst composition of claim 1, wherein
the nanostructure catalyst composition comprises less than 10% or more than 90% by mole of C.

11. The nanostructure catalyst composition of claim 1, wherein
the nanostructures of the nanostructure catalyst composition each independently is from about 1 nm to about 3000 nm in length, width or height, or the nanostructures each independently has an aspect ratio of from about 1 to about 20, from about 1 to about 10, or from about 2 to about 8.

12. The nanostructure catalyst composition of claim 1, wherein
the nanostructures each independently has a shape of spherical, spike, flake, needle, grass, cylindrical, polyhedral, 3D cone, cuboidal, sheet, hemispherical, irregular 3D shape, porous structure or any combinations thereof.

13. The nanostructure catalyst composition of claim 1, wherein
multiple nanostructures are arranged in a patterned configuration, in a plurality of layers, on a substrate, or randomly dispersed in a medium.

14. A method for producing organic molecules having at least two carbon atoms chained together by the reaction of a hydrogen-containing source, a carbon-containing source and an optional nitrogen-containing source in the presence of a nanostructure catalyst composition according to claim 1.

15. The method of claim 14, wherein
the organic molecules comprise saturated, unsaturated and aromatic hydrocarbons, carbohydrates, amino acids, polymers, or a combination thereof.

16. The method of claim 15, wherein
the organic molecules comprise linear saturated hydrocarbons having 20 carbon atoms or less when the catalytic property provider is selected from the group consisting of Co, Mn or combination thereof.

17. The method of claim 15, wherein
the organic molecules comprise linear saturated hydrocarbons having 20 carbon atoms or more when the catalytic property provider is Fe.

18. The method of claim 15, wherein
the organic molecules comprise linear saturated hydrocarbons having 3 carbon atoms or less when the catalytic property provider is selected from the group consisting of Ni, Cu or combination thereof.

19. The method of claim 15, wherein
the organic molecules comprise linear and branched, saturated and unsaturated hydrocarbons having 5 to 10 carbon atoms when the catalytic property provider is selected from the group consisting of Ru, Rh, Pd, Os, Ir, La, Ce or any combination thereof.

20. The method of claim 14, wherein
the reaction is initiated by light irradiation or initiated by heat.

21. The method of claim 14, wherein
the reaction is progressed under a temperature between about 50° C. and about 800° C.

22. The method of claim 14, wherein
the carbon-containing source comprises CO₂ or CO, and
the hydrogen-containing source comprises water.
